(19) United States
(12) Patent Application Publication
(10) Pub. No.: US 2005/0288618 A1
(43) Pub. Date: Dec. 29, 2005

(54) Myocardial treatment apparatus and method

(75) Inventors: Mark L. Jenson, Greenfield, MN (US); William J. Drasler, Minnetonka, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SciMed Life Systems, Inc.

(21) Appl. No.: 10/877,331

(22) Filed: Jun. 24, 2004

(51) Int. Cl. A61M 39/00; A61F 2/06

(52) U.S. Cl. 604/8; 128/898; 623/1.41

(57) ABSTRACT

Apparatus and methods for forming a conduit through an infarcted region where the conduit is in fluid communication with a supply of oxygenated blood and the venous vasculature and flowing oxygenated blood from the supply of oxygenated blood through the conduit and to the venous vasculature.

### FIELD OF THE INVENTION

The present invention relates generally to methods and apparatus for treating the myocardium. More specifically, the present invention pertains to methods and apparatus for treating infarcted regions of the myocardium.

### BACKGROUND OF THE INVENTION

Treatment of myocardial infarction (MI) has progressed significantly, particularly in rapidly opening occluded coronary arteries with thombolytic agents, balloon catheters, and other devices, in certain acute myocardial infarctions (AMIs) to minimize the amount of tissue death and minimize the size of the infarcted region. In many cases, however, a vessel is not opened rapidly enough to avoid a significant infarct. Depending on the size of the infarct, myocardial infarction can lead to heart failure due to the decreased ability of the heart to properly pump blood. In addition to decreased (or no) cardiac muscle function in the infarcted area and replacement with collagenous scar tissue, stretching and dilation of the heart can occur, further increasing the stress on the myocardium and exacerbating the situation, leading to progression of heart failure. Various methods have been used to intervene in these patients, including medications to decrease vascular resistance and reduce cardiac workload, stimulating cardiac muscle, improving perfusion in the myocardium, use of counter pulsation devices, surgical resection of infarcted or enlarged myocardium, injection of myocytes, stem cells or other cells and other methods and devices.

Portions of the myocardium which are deprived of perfusion for periods of more than an hour or two will have cell death of the cardiomyocytes in those regions. Some limited perfusion by collateral vessels may reduce cell death, particularly in the border areas at the margins of the infarct area, but such collateral circulation is typically insufficient to support a normally functioning myocardium resulting in poorly functioning or dead myocardium. If a vessel supplying an infarcted area is opened with angioplasty or coronary artery bypass, the vessel will likely not remain patent because there are very few cells in the infarcted region, and therefore no need to for vasculature of any significant size. Transmyocardial revascularization can produce openings for blood flow, but will quickly occlude by thrombosis and likewise have no reason to result in a stable vessel since there are few cells to perfuse. Endothelium, responding to flow of nutrients, plasma or blood will not respond by angiogenesis and creation of significant vasculature. Yet any repopulation of the infarcted area by functioning cells cannot occur because the area has no blood supply. Therefore, only minimal recovery and repopulation at the extreme margins of the infarcted zone can occur. The resulting continued deterioration to heart failure is then the likely outcome. The present invention provides a treatment for infarcted myocardium so that continued progression to heart failure can be delayed or avoided.

### SUMMARY OF THE INVENTION

Generally, the present invention relates to methods and apparatus for treating the myocardium. More specifically, the present invention pertains to methods and apparatus for treating infarcted regions of the myocardium.

The present invention relates to a method which is particularly applicable to the human heart having an infarcted region and a venous vasculature in fluid communication with a right atrium of the heart. A preferred method includes forming a conduit through the infarcted region where the conduit is in fluid communication with a supply of oxygenated blood and the venous vasculature and flowing oxygenated blood from the supply of oxygenated blood through the conduit and the venous vasculature to the right atrium. The increased blood flow through the infarcted region facilitates healing and revascularization of the infarcted region.

The above summary of the present invention is not intended to describe each disclosed embodiment or every implementation of the present invention. The Figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIGS. 1A and 1B are partial surface views of a human heart illustrating the cardiac venous vasculature;

FIG. 2 is a sectional view of the myocardium, showing certain components of the cardiac venous system;

FIG. 3 is a sectional view of the myocardium, showing certain components of the cardiac venous system;

FIG. 4A is a sectional view of a human heart showing the placement of an embodiment of the invention;

FIG. 4B is an enlarged sectional view of a portion of a human heart showing the placement of an embodiment of the invention;

FIG. 4C is a sectional view of a portion of a human heart showing the placement of another embodiment of the invention;

FIG. 5 a sectional view of the myocardium, showing placement of certain components of the embodiment shown in FIG. 4;

FIG. 6A is a plan view of a conduit creation device generally;

FIGS. 6B-6H are views of exemplary conduit creation devices as shown in FIG. 6;

FIG. 7 is a perspective view of the conduit through an infarcted region of the myocardium of FIG. 4; and

FIG. 8 is a perspective view of the conduit of FIG. 7 including an illustrative stent within the conduit.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

The present invention applies to healing of tissue after an episode of ischemia which has resulted in tissue death. More particularly, the present invention relates to treatment of infarcted regions of myocardium or hypoperfused tissue. The present invention has particular advantages in treating myocardial tissue, but has applicability in treating other ischemic infarcts as well. The invention includes methods of perfusing infarcted regions, methods of delivering cells, growth factors, and other biological materials, methods of maintaining viability of cells in infarcted regions and fabrication methods. The invention provides apparatus and methods for facilitating healing and maintaining viability of healing tissue in previously infarcted areas, and can be utilized early or late after an infarct. Additionally, regions of the myocardium that are generally scarred due to possible prior myocardial infarctions can also be treated using the device and methods of the present invention.

The present invention provides a more complete treatment than prior treatments. If a patient presents during an AMI due to acute occlusion of coronary arteries, interventional methods such as thrombolytic or other medication and angioplasty or other devices can be used to reopen the affected arteries and limit the resulting injury to the myocardium. In most cases, however, myocardial injury cannot be eliminated. In addition, many patients present too long after the arterial occlusion, so that a significant portion of myocardium is dead. The present invention provides a treatment in the post-MI period for enhancing the myocardial healing and recovering myocardial function in the previously infarcted region. Late treatments exist for patients with heart failure, although they are not optimal. Preventing the progression to heart failure can offer a great benefit. The present invention therefore fills a void in the treatment approaches so that a patient can now be treated in the AMI, post-MI, and heart failure stages of disease.

The myocardial arteriovenous (AV) shunts created can be combined with other therapeutic means using devices and technology according to the present invention to provide a superior treatment over prior art. Combination therapy can include infusion of growth factors, medications, or other materials into the AV shunts or the surrounding myocardium using medical devices, placement of myocytes, cardiomyocytes, stem cells, gene transfer agents, or other biological materials in the AV shunts or the surrounding myocardium using placement devices, use of biologic materials (such as, for example, submucosal tissue, elastin, collagen, PLLA, PGA, or other biological or bio-reabsorbable materials) as stents and/or material delivery devices in the AV shunts using medical devices, medical delivery devices and technology.

Many competing technologies such as injection of cells, creation of revascularization channels, and so forth are believed insufficient because they require enhanced perfusion to maintain the viability of those cells as well as the viability of the tissue being treated, but do not have the enhanced perfusion provided by the present invention.

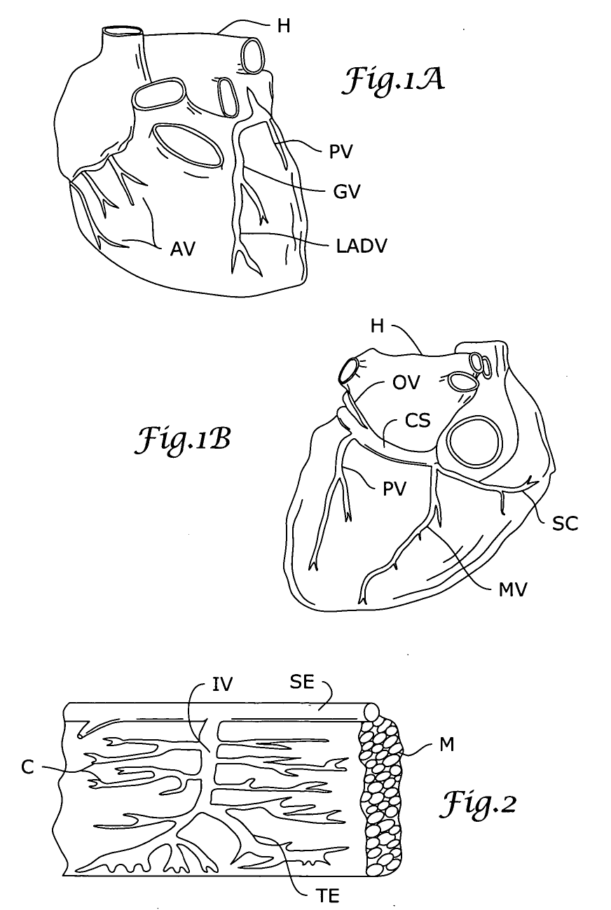

Referring to FIGS. 1A, 1B and 2, the coronary venous vasculature of a human heart H and a model of the myocardial veins, respectively, are described. The venous system includes the coronary sinus CS that provides drainage for the great cardiac vein GV, the left anterior descending cardiac vein LADV, the middle cardiac vein MV, the oblique vein of the left atrium OV, the posterior vein of the left ventricle PV and small cardiac vein SC. Deoxygenated blood flowing into coronary sinus CS exits via the coronary ostium CO into the right atrium. The venous system further includes the anterior cardiac veins AV that drain directly into the right atrium. Representative cardiac veins are shown for illustrative purposes only. It is recognized that other veins exist and can vary between individual patients.

With respect to FIG. 2, myocardium M includes a lattice of capillaries C that drain deoxygenated blood into the intramyocardial veins IV. As is known, the capillaries connect to the arterioles and the coronary artery system (not shown). From the intramyocardial veins IV, a fraction of the blood drains into the cardiac veins via the subepicardial veins SE, while the remainder drains through the Thebesian veins TE directly into the atrial and ventricular cavities. It has been reported in healthy human hearts that approximately 70% of the deoxygenated blood is drained through the coronary sinus, while the remaining 30% is drained in about equal proportions into the left and right atria and ventricles via the lymphatic system and the Thebesian veins. It has likewise been reported that when individual components of the venous system (i.e., the coronary sinus, lymphatic system and Thebesian veins) are occluded, the flow redistributes itself through the remaining unoccluded channels.

The coronary arteries are formed of resilient tissue fibers that withstand the pressures typically generated in the left ventricle during cardiac systole, typically up to a peak pressure of about 120 mm Hg, but can vary depending on an individual patient's blood pressure. By contrast, the tissue fibers of the cardiac venous system are much less resilient than those of the coronary arterial system, with pressures in the coronary sinus generally not exceeding 6-10 mm Hg.

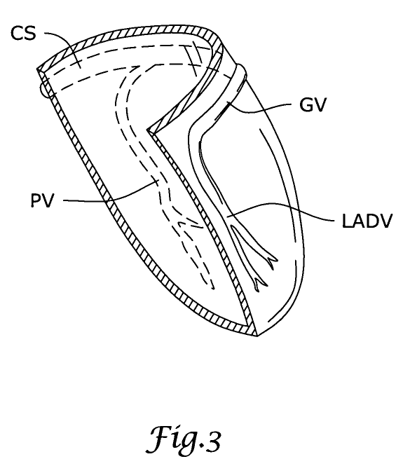

In FIG. 3 the relative positions of portions of the coronary venous vasculature are shown with respect to the left ventricle, i.e., those vessels disposed on the epicardium directly overlying the left ventricle. More specifically, portions of the coronary sinus CS, the great cardiac vein GV, the left anterior descending cardiac vein LADV, and posterior vein of the left ventricle PV, overlie the left ventricle. The spatial relationships of the coronary sinus and veins depicted in FIG. 3 are intended to be merely illustrative, since normal hearts can show a considerable degree of variation.

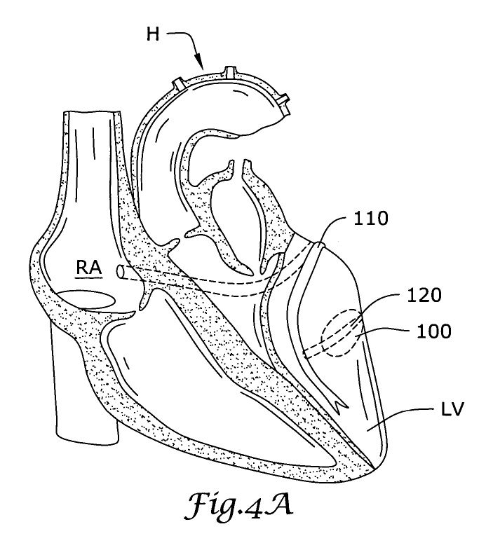

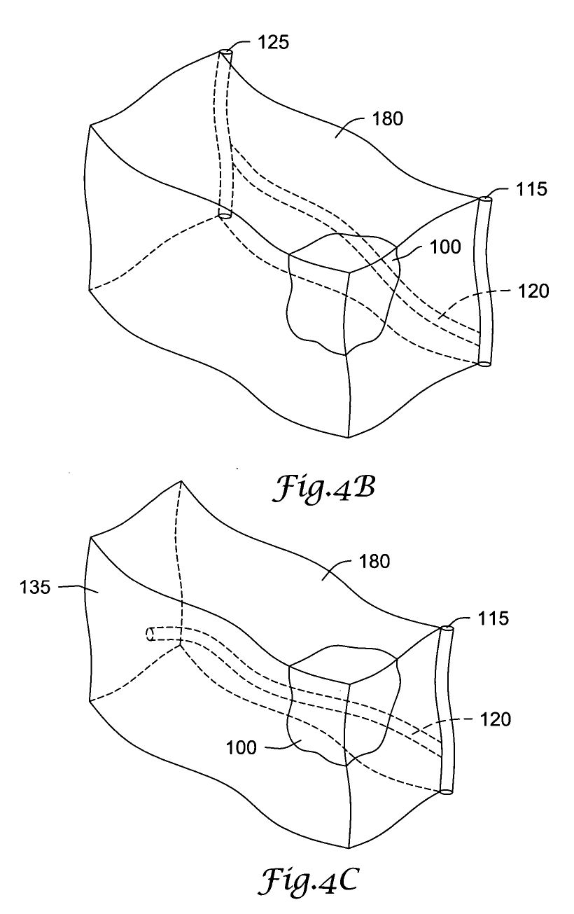

Referring now to FIG. 4A, the human heart treated with the method and apparatus of the present invention is described. FIG. 4A depicts human heart H partly in cross-section, where the apparatus of the present invention has been deployed in accordance with the methods described hereinafter. Human heart H includes a venous vasculature 110 in fluid communication with a right atrium RA. The venous vasculature 110 overlays a portion of the left ventricle LV. An infarcted region 100 overlays a portion of the left ventricle LV.

A conduit or AV shunt 120 is disposed over or within at least a portion of the infarcted region 100 and in fluid communication with the venous vasculature 110.

A preferred embodiment of the present invention includes a percutaneous AV shunt or conduit 120 which can be guided into epicardial veins in or adjacent to the infarct zone 100. The AV shunt or conduit 120 is passed out of the venous vasculature 110, through the infarcted myocardium 100, and into the ventricle LV. Preferably, a long path through the infarct zone is created to maximize the region of infarcted myocardium surface area of enhanced perfusion. Optionally, as shown in FIG. 4B, the AV shunt or conduit 120 can be directed to a nearby epicardial coronary artery 125. It is desirable, but not necessary to maintain a primarily subendocardial path. A subendocardial path is preferred since the subendocardial region is typically the primary infarct site. Safely directing the AV shunt or conduit 120 to the ventricular space is simpler than directing the AV shunt or conduit 120 to a coronary artery. An AV shunt path or conduit 120 of approximately 1 mm to 4 mm diameter range is preferred to obtain sufficient oxygenated blood flow through the infarct region 100 to remain patent for at least several weeks, but small enough to avoid undue strain on the heart due to the AV shunt or conduit 120. Oxygenated blood flow rates through the AV shunt or conduit 120 of approximately 50 mL/min to 500 mL/min are preferred.

FIG. 4B shows AV shunt or conduit 120 passing through or near infarction region 100. AV shunt or conduit 120 may extend from coronary artery 125 to epicardial vein 115. Alternatively, FIG. 4C shows the AV shunt or conduit 120 may extend from the endocardial surface 135 of left ventricle LV to epicardial vein 115. Although an epicardial vein is shown, the vein could be located within the myocardium 180.

Steerable devices may be used, or separate guidance apparatus may be utilized, to guide the AV shunt or conduit 120 to the delivery site. Mechanical, electrical, or magnetic guidance or steering may be used. Imaging features and imaging apparatus maybe utilized, including radiograph, MRI, CT, ultrasound, or other mechanisms and methods.

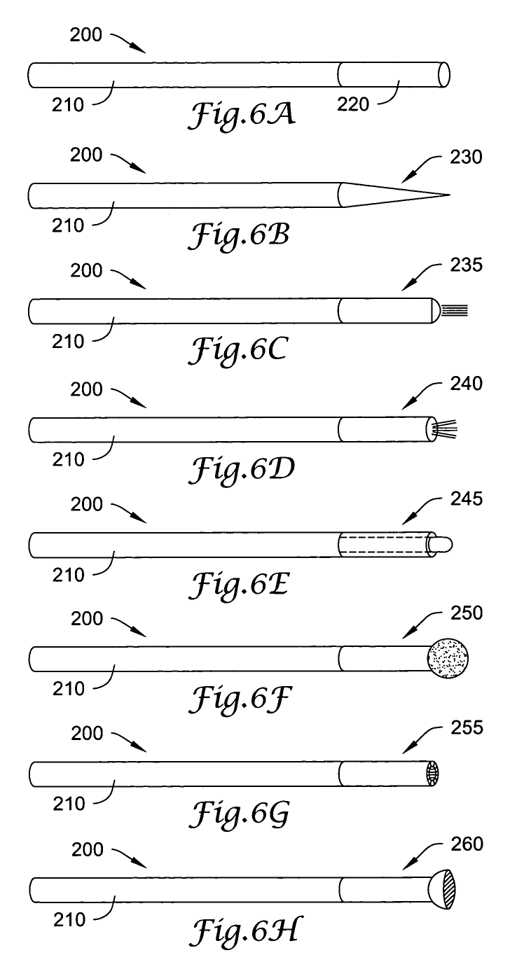

The invention preferably includes shunt or conduit creation device 200. The shunt or conduit creation device 200 may be used to facilitate delivery of the AV shunt or conduit 120 to the infarct region 100 in a number of ways, including, for example, intravascularly or surgically. FIG. 6A illustrates generally one such shunt or conduit creation device 200. Shunt or conduit creation device 200 may include a directing means 210 and a conduit forming means 220. The shunt or conduit creation device can be delivered intravascularly in a number of ways, for example, via a guiding catheter, a guide wire, or the like. As shown in FIG. 6B, the shunt or conduit creation device may include a simple piercing apparatus with a piercing needle, a stylet and a dilator 230. Alternatively, a thermal catheter can be used to create the shunt or conduit 120 path through the infarct region 100. Still other alternatives of shunt or conduit creation device 200 as shown in FIGS. 6C-6H include, for example, laser 235, fluid jet 240, ultrasound vibration 245, rotating burr 250, rotating hole cutter 255, rotating milling cutter 260, or other catheter device.

Surgical versions of the present invention (that is, not intended for percutaneous or intravascular access procedures, but for open surgical access procedures) can be utilized, and may be particularly useful in certain anatomical situations in which percutaneous access to the desired tissue region is difficult or impossible.

The invention may include an apparatus for harvesting cells from the myocardium; the apparatus may include cutting means such as a needle or scraper, and may include suction means or a holding chamber to allow withdrawal of the cells from the body.

In the period after creation of the myocardial AV shunt or conduit 120, tissue may migrate from neighboring areas or from remote areas via the blood to repopulate the infarct zone 100. Cellular proliferation in the AV shunt or conduit 120 path may progress to eventually occlude the AV shunt or conduit 120 over time, during the healing period, new arterial and venous circulation paths may form to perfuse the new tissue, so that occlusion of the AV shunt or conduit 120 is acceptable and may be preferred. It is preferred to maintain sufficient perfusion by the AV shunt or conduit 120 so that significant tissue regeneration in the areas adjacent to the AV shunt or conduit 120 can occur before the AV shunt or conduit 120 occludes.

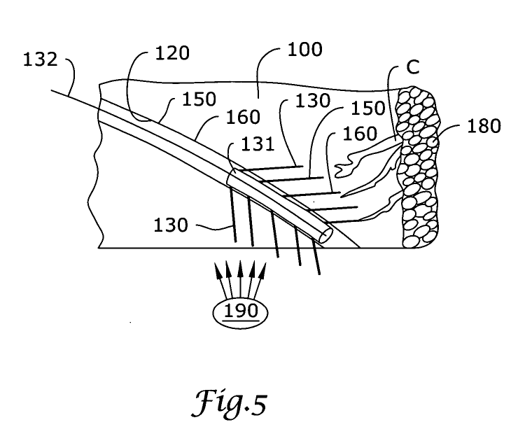

FIG. 5 shows a section of infarcted myocardium 100 including an embodiment of the invention. FIG. 5 shows an infarcted region 100 through at least a portion of the myocardium 180. The AV shunt or conduit 120 is disposed near or through a portion of the infarcted region 100 in fluid communication with a source of oxygenated blood 190. A passageway 130 can be formed through at least a portion of the infarcted region 100 in fluid communication with a source of oxygenated blood 190 and the AV shunt or conduit 120.

The passageway 130 or AV shunt or conduit 120 may extend through the myocardium 180 of the left ventricle LV (source of oxygenated blood) or may extend through a portion of the myocardium 180 to a capillary C, which provides an oxygenated blood source 190. The conduit or AV shunt 120 may be in fluid communication with 1, 2, 3, 4, 10, 20, 30 or more passageways 130 that may provide oxygenated blood flow through the infarcted region 100 of the myocardium 180. The passageways 130 may be any size adequate to provide the desired blood flow through the infarcted region 100. The passageways 130 may be up to approximately 3 mm, but typically less than approximately 1 mm in diameter. The conduit or AV shunt 120 can be formed by tunneling through the myocardium 180 or made of any biocompatible material including grafted tissue. Both the AV shunt or conduit 120 and the passageway 130 fluid flow rate may vary over preselected time duration. The flow rate in AV shunt or conduit 120 may decrease to zero over a time period, for example, one hour, one day, one month, two months, or three months. For example, a stent-like structure or metallic ring structure may be designed to heat due to application of an extensive electromagnetic filed resonating in a controlled occlusion. The flow rate in passageways 130 may increase over a time period of three days to three months, for example, as the myocardium 180 heals. The AV shunt or conduit 120 or passageways 130 may be formed from tunneling through the myocardium 180 with a device or the AV shunt or conduit 120 may be formed from stents, vascular grafts, stent-grafts, and other conduits such as TMR passages. The conduit or conduit covering may additionally contain medicinal drugs.

The invention may include the use of a perforation device 131 used to create multiple holes, long channels or passageways 130 in the myocardium 180 infarct region 100 adjacent the shunt or conduit 120; these holes or passageways 130 can be used as sites for delivery or retention of cells, medications, or other materials near the blood supply of the AV shunt or conduit 120 but minimizing any rapid washout that otherwise could occur. The created multiple holes, long channels or passageways 130 in the myocardium 180 infarct region 100 may occlude with thrombus immediately, but would provide channels to facilitate new tissue ingrowths, formation of new blood vessels, and healing of a larger region than that immediately adjacent the AV shunt or conduit 120. The perforation device 131 may be the same device as the AV shunt or conduit creation device 200 previously described or a simple stylet or needle could be used, or the perforation device 131 can create multiple passageways 130 with similar mechanisms.

The perforation device 131 can be delivered to the treatment site via any delivery means such as, for example, a guide wire 132. The perforation device 131 can remain in situ or can be withdrawn from the body using the guide wire 132.

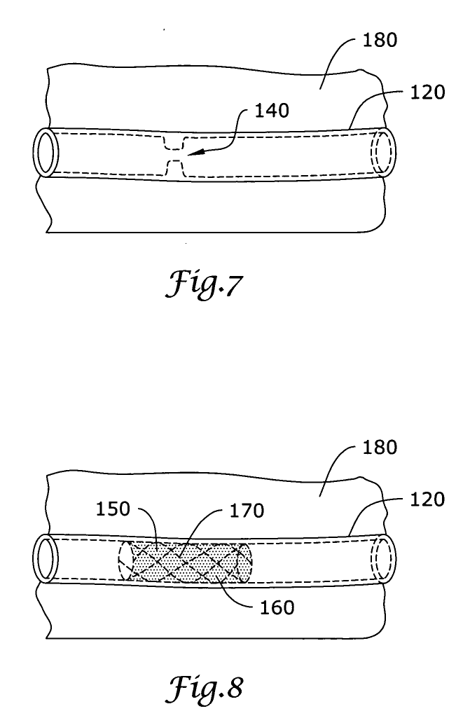

Referring now to FIG. 7, the AV shunt or conduit 120 is further described. An optional restriction or occlusion 140 in the AV shunt or conduit 120 can be formed to control the rate of blood flow and pressure through the AV shunt or conduit 120; the restriction 140 can be formed by forming the tissue with a balloon, thermal, chemical, cryothermal, or other device or can be formed by implanting a device from a cardiac vein or other low-pressure region to a ventricle chamber or atrium chamber or coronary artery or other high-pressure region, or passage of a device from a high-pressure region to a low-pressure region, or a combination meeting in between the high- and low-pressure regions. The AV shunt or conduit 120 may include a restriction or a partial, adjustable, or time-varying occlusion 140 which controls the fluid flow rate through AV shunt or conduit 120 and also provides both pressure on the oxygenated blood flowing through the passageways 130 and conduit 120 allowing the oxygenated blood to perfuse into infarcted regions 100 of the myocardium 180. The restriction or occlusion 140 also provides that the blood flow downstream of the restriction or occlusion has a sufficiently low enough pressure as to not damage the venous vasculature 110. The venous vasculature may have a pressure lower than the arterial pressure.

Now referring to FIG. 8, a stent may be placed within the AV shunt or conduit 120 or within the passageways 130. The stent 170 may be any commercial available stent, such as, for example, a stent 170 of the type described in U.S. Pat. No. 4,655,771. Stent 170 may be, for example, a woven mesh structure covered with a polyurethane coating. The stent 170 may be transformable between a reduced diameter and an expanded diameter.

The invention can include a stent 170 to maintain patency of the shunt or conduit 120 or passageways 130 for a time, a narrowing or occlusion 140 within the shunt 120 or stent 170 could be used to control the blood flow rate through the shunt 120, or the diameter of the shunt 120 could be made or adjusted to obtain an appropriate flow rate. A stent 170 can be used to control or maintain the diameter of the AV shunt or conduit 120 or passageways 130.

The stent 170 can be balloon expandable, self-expanding, or can be formed in place, and can be durable or bioresorbable or a combination, and can have cells, medications or other biological materials incorporated or attached. The stent may be metallic, polymeric or a combination of materials, and may be durable, biodegradable or bioabsorbable and the stent 170 may include a medicament 150 or biological material 160 applied thereon. A porous covering on the stent 170 or AV shunt 120 could be used, a cylindrical covering, or a wrapped sheet, band, or tape and can be durable such as of PTFE or silicone, or can be bio-reabsorbable such as PGA, PLLA, or gelatin, or a modified or unmodified elastin, collagen, or other protein.

A stent 170 or stent delivery device can be utilized to deliver chemolytic agents, chemotactic agents, growth factors, cells, myocytes, cardiomyocytes, stem cells, gene transfer agents, coagulation modification agents, angiogenic agents, imaging enhancing agents, or other material(s) in the stent 170, AV shunt or conduit 120 or the myocardium 180 in conjunction with the present invention. Autologous cardiomyocytes or other cells can be obtained using a harvesting device, for transfer in the infarct tissue 100 adjacent to the AV shunt or conduit 120 or passageways 130 to seed the area with cells.

A stent 170 comprising biological material (elastin, collagen, etc.) can be utilized to provide a scaffold for tissue growth. A stent 170 can be used as a material delivery device, to deliver medications, cells or other materials to enhance tissue growth and healing. An implantable device such as a porous material can be implanted in the infarct area 100 with the AV shunt 120 or in the adjacent tissue to similarly be a material delivery device but separate from any stent 170. A stent 170 or other implanted device comprising bioresorbable material can be utilized so that the device disappears after it is no longer needed, resulting in a more normal myocardial structure. Material to inhibit scarring, inhibit fibroblast migration and proliferation and collagen synthesis, promote cardiomyocyte migration and proliferation, promote smooth muscle cell migration and proliferation, or promote endothelial cell migration and proliferation may be delivered to the infarct site 100 via a delivery device such as, for example the stent 170. The stent 170 or implanted device may comprise material which dissolves or reabsorbs over time to reveal or release pro-thrombotic material or agents, providing for occlusion of the AV shunt after a day, a week or 1, 2, 3, or 4 months, for example, when it is no longer needed. Alternatively, the stent 170 or implanted device can incorporate occlusion means 140 which can provide for occlusion of the AV shunt 120. This occlusion means 140 can include external activation means such as by magnetic or electric field, ultrasound, radiation, or other energy transfer, or enzymatic or other chemical means to effect occlusion. Preferably, such occlusion is accomplished non-invasively, although intravascular catheter means for occlusion can be utilized as well.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

1. A method for treating a heart having an infarcted region and a venous vasculature in fluid communication with a right atrium of the heart comprising the steps of:
providing a conduit creation apparatus;
forming a conduit through the infarcted region wherein the conduit is in fluid communication with a supply of oxygenated blood and the venous vasculature; and
carrying oxygenated blood from the supply of oxygenated blood through the infarcted region to the venous vasculature.

2. The method according to claim 1, wherein the step of forming a conduit further comprises forming a plurality of passageways through the infarcted region.

3. The method according to claim 2, wherein the passageways are in fluid communication with the supply of oxygenated blood.

4. The method according to claim 1, wherein the supply of oxygenated blood is a left ventricle.

5. The method according to claim 1, wherein the supply of oxygenated blood is an artery.

6. The method according to claim 1, wherein the venous vasculature is in fluid communication with a coronary sinus.

7. The method according to claim 1, wherein the conduit includes an occlusion for obtaining a pre-selected fluid flow rate through the conduit.

8. The method according to claim 1, wherein the conduit includes a restriction for obtaining a pre-selected fluid flow rate through the conduit.

9. The method according to claim 1, wherein the conduit has a fluid flow rate that decreases over a pre-selected time duration.

10. The method according to claim 1, wherein the conduit has a fluid flow rate from 50 to 500 mL/min.

11. The method according to claim 1, further comprising disposing a medicament on the conduit.

12. The method according to claim 2, further comprising disposing a medicament within the passageways.

13. The method according to claim 11, wherein the medicament is a chemotactic agent, coagulation modification agent, gene transfer agent, growth factor or angiogenic agent.

14. The method according to claim 12, wherein the medicament is a chemotactic agent, coagulation modification agent, gene transfer agent, growth factor or angiogenic agent.

15. The method according to claim 1, further comprising disposing a biological material on the conduit.

16. The method according to claim 2, further comprising disposing a biological material on the passageways.

17. The method according to claim 15, wherein the biological material is a myocyte, cardiomyocytes, or stem cell.

18. The method according to claim 16, wherein the biological material is a myocyte, cardiomyocytes, or stem cell.

19. The method according to claim 1, further comprising disposing a stent within the conduit.

20. The method according to claim 19, further comprising disposing a medicament on the stent.

21. The method according to claim 19, wherein the medicament is a chemotactic agent, coagulation modification agent, gene transfer agent, growth factor or angiogenic agent.

22. The method according to claim 19, further comprising disposing a biological material on the stent.

23. The method according to claim 22, wherein the biological material is a myocyte, cardiomyocytes, or stem cell.

24. The method according to claim 23, wherein the stent is bio-absorbable.

25. The method according to claim 1, wherein the venous vasculature is at a pressure lower than an arterial pressure.

26. A method for treating hypoperfused or infarcted tissue comprising the steps of:
providing a conduit creation apparatus;
forming a conduit through or adjacent to the hypoperfused or infarcted region from a source of oxygenated blood to a source of deoxygenated blood; and
using the conduit to carry a flow of oxygenated blood through or adjacent to the hypoperfused or infarcted tissue.

27. The method according to claim 26, wherein the source of oxygentated blood is an artery.

28. The method according to claim 26, wherein the source of deoxygenated blood is an epicardial vein.

29. The method according to claim 26, wherein the conduit creation apparatus includes a conduit forming means and a directing means.

30. The method according to claim 26, further comprising providing a perforation forming means, the perforation forming means forming at least one perforation.

31. The method according to claim 30, wherein the perforation forming means forms a plurality of perforations.

32. A device for creating a conduit from an oxygenated blood supply to a vein, comprising;
an expandable element having a deliverable position having a first diameter and an expanded position having a second diameter larger than the first diameter.

33. The device in claim 32, wherein the expandable element is a stent.

34. The device in claim 33, wherein the stent is covered by a covering.

35. The device in claim 33, wherein the stent includes a medicament.

36. The device in claim 34, wherein the covering includes a medicament.

37. The device in claim 33, wherein the stent is bio-absorbable.

38. The device in claim 33, wherein the stent includes a flow reducing area.
